(19) United States
(12) Patent Application Publication
(10) Pub. No.: US 2024/0099606 A1
(43) Pub. Date: Mar. 28, 2024

(54) HEAD-MOUNTED EMITTER ASSEMBLY

(71) Applicant: Biosense Webster (Israel) Ltd., Yokneam (IL)

(72) Inventors: Meir Bar-Tal, Haifa (IL); Shmuel Auerbach, Kerem Maharal (IL); Barak Nadav, Haifa (IL); George Gusein, Acre (IL); Erez Mardo, Mikhmoret (IL)

(21) Appl. No.: 18/243,146

(22) Filed: Sep. 7, 2023

Related U.S. Application Data
(60) Provisional application No. 63/407,209, filed on Sep. 16, 2022.

(51) Int. Cl. A61B 5/06; A61B 34/20

(52) U.S. Cl. CPC A61B 5/062; A61B 34/20; A61B 2034/2051; A61B 2034/2072

(57) ABSTRACT

An apparatus includes a frame and a plurality of magnetic field generators. The frame is configured to be secured to a patient's head. The frame includes a plurality of housings. Each housing of the plurality of housings is configured to securely engage a corresponding portion of the patient's head. The plurality of magnetic field generators is operable to generate a magnetic field around at least a portion of the patient's head. Each magnetic field generator of the plurality of magnetic field generators is securely retained by a corresponding housing of the plurality of housings.

### PRIORITY

This application claims priority to U.S. Provisional Pat. App. No. 63/407,209, entitled “Head-Mounted Emitter Assembly,” filed Sep. 16, 2022, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, now abandoned, the disclosure of which is incorporated by reference herein. An example of such a system is the Relieva® Spin Balloon Sinuplasty™ System by Acclarent, Inc. of Irvine, California.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. In this manner, the surgeon is able to know the precise position of each sensor equipped instrument by viewing the video monitor even if the surgeon is unable to directly visualize the instrument itself at its current location within the body.

An example of an electromagnetic IGS systems that may be used in ENT and sinus surgery is TRUDI ° Navigation System from Acclarent, of Irvine, California. When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. As a result, IGS systems may be particularly useful during performance of FESS, balloon sinuplasty, and/or other ENT procedures where anatomical landmarks are not present or are difficult to visualize endoscopically. Another example is the use of IGS for placement of cochlear implants, during which the patient's ear is facing the surgeon during the procedure.

One function that may be performed by an IGS system is obtaining a reference point that can be used to correlate various preoperatively obtained images with a patient's actual position during a procedure. This act may be referred to as patient registration. Patient registration is conventionally performed by using a positionally tracked instrument (e.g., a guidewire whose tip position may be detected in three-dimensional space) to trace the area of a patient that will be affected by the procedure. For example, in the case of a balloon sinuplasty or other ENT procedure, a positionally tracked guidewire or other tool may be used to trace or touch one or more positions on a patient's face. At each touch point, a positional tracking system will register that point in three-dimensional space and, using a number of registered points, determine the position of the affected area in three-dimensional space. Once the affected area is fully mapped or registered, it can be correlated with preoperative images in order to provide a seamless IGS experience across varying types of preoperative images during the performance of the procedure. Performing patient registration in this manner is both time consuming and error prone, due to the number of touch points required for some procedures and the relative inaccuracy of pressing a flexible guidewire tip against the non-rigid surface of a patient's face.

It may be desirable to provide features that further facilitate the use of an IGS navigation system and associated components in ENT procedures and other medical procedures. While several systems and methods have been made and used with respect to IGS and ENT surgery, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a sinus surgery navigation system being used on a patient seated in an example of a medical procedure chair;

FIG. 2 depicts a front perspective view of an example of an electromagnetic field emitter assembly secured to a front region of a patient's head;

FIG. 3 depicts a cross-sectional view of the electromagnetic field emitter assembly of FIG. 2, with a patient's head shown in phantom to reveal an adjustable strap of the electromagnetic field emitter assembly; and

FIG. 4 depicts a front perspective view of an example of an electromagnetic field emitter assembly secured to a side region of a patient's head.

### DETAILED DESCRIPTION

The inventors have conceived of novel technology that, for the purpose of illustration, is disclosed herein as applied in the context of image guided surgery. While the disclosed applications of the inventors' technology satisfy a long-felt but unmet need in the art of image guided surgery, it should be understood that the inventors' technology is not limited to being implemented in the precise manners set forth herein, but could be implemented in other manners without undue experimentation by those of ordinary skill in the art in light of this disclosure. Accordingly, the examples set forth herein should be understood as being illustrative only and should not be treated as limiting.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Example of an Image Guided Surgery Navigation System

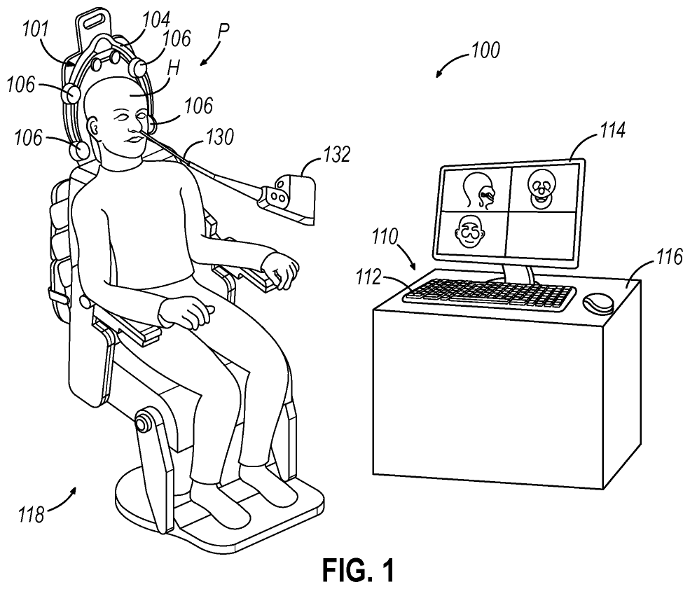

FIG. 1 shows an example of an IGS navigation system (100) enabling an ENT procedure to be performed using image guidance. In some instances, IGS navigation system (100) is used during a procedure where a dilation instrument assembly (not shown) is used to dilate the ostium of a paranasal sinus; or to dilate some other anatomical passageway (e.g., within the ear, nose, or throat, etc.). In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein; U.S. Pat. No. 10,362,965, entitled “System and Method to Map Structures of Nasal Cavity,” issued Jul. 30, 2019; and U.S. Pat. Pub. No. 2011/0060214, entitled “Systems and Methods for Performing Image Guided Procedures within the Ear, Nose, Throat and Paranasal Sinuses,” published Mar. 10, 2011, now abandoned, the disclosure of which is incorporated by reference herein.

IGS navigation system (100) of the present example comprises a field generator assembly (101), which comprises set of magnetic field generators (106) that are integrated into a horseshoe-shaped frame (104). Field generators (106) are operable to generate alternating magnetic fields of different frequencies around the head of the patient. Field generators (106) thereby enable tracking of the position of a navigation guidewire (130) that is inserted into the head of the patient. Various suitable components that may be used to form and drive field generators (106) will be apparent to those of ordinary skill in the art in view of the teachings herein.

In the present example, frame (104) is mounted to a chair (118), with the patient (P) being seated in the chair (118) such that frame (104) is located adjacent to the head (H) of the patient (P). By way of example only, chair (118) and/or field generator assembly (101) may be configured and operable in accordance with at least some of the teachings of U.S. Pat. No. 10,561,370, entitled “Apparatus to Secure Field Generating Device to Chair,” issued Feb. 18, 2020, the disclosure of which is incorporated by reference herein.

IGS navigation system (100) of the present example further comprises a processor (110), which controls field generators (106) and other elements of IGS navigation system (100). For instance, processor (110) is operable to drive field generators (106) to generate electromagnetic fields; and process signals from navigation guidewire (130) to determine the location of a sensor in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

A coupling unit (132) is secured to the proximal end of a navigation guidewire (130). Coupling unit (132) of this example is configured to provide wireless communication of data and other signals between console (116) and navigation guidewire (130). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Navigation guidewire (130) may be used in a dilation instrument (not shown). Navigation guidewire (130) includes a position sensor (not shown) that is responsive to alternating electromagnetic fields generated by field generators (106). In the present example, the sensor of navigation guidewire (130) comprises at least one coil at the distal end of navigation guidewire (130). When such a coil is positioned within an alternating electromagnetic field generated by field generators (106), the alternating electromagnetic field may generate electrical current in the coil, and this electrical current may be communicated along the electrical conduit(s) in navigation guidewire (130) and further to processor (110) via coupling unit (132). This phenomenon may enable IGS navigation system (100) to determine the real-time location of the distal end of navigation guidewire (130) within a three-dimensional space (i.e., within the head (H) of the patient (P)). To accomplish this, processor (110) executes an algorithm to calculate location coordinates of the distal end of navigation guidewire (130) from the position related signals of the coil(s) in navigation guidewire (130). While navigation guidewire (130) includes a position sensor in the present example, various other kinds of instrumentation may include one or more position sensors. The teachings herein may therefore be readily applied to the context of any kind of instrumentation including one or more position sensors that cooperate with field generators (106) to generate signals indicating the real-time position of the sensor equipped instrumentation.

Processor (110) uses software stored in a memory of processor (110) to calibrate and operate system (100). Such operation includes driving field generators (106), processing data from navigation guidewire (130), processing data from operating controls (112), and driving display screen (114). Processor (110) is further operable to provide video in real time via display screen (114), showing the position of the distal end of navigation guidewire (130) in relation to a video camera image of the head (H) of the patient (P), a CT scan image of the head (H) of the patient (P), and/or a computer-generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the head (H) of the patient (P), such as navigation guidewire (130), such that the operator may view the virtual rendering of the instrument at its actual location in real time. By way of example only, display screen (114) may provide images in accordance with at least some of the teachings of U.S. Pat. No. 10,463,242, entitled “Guidewire Navigation for Sinuplasty,” issued Nov. 5, 2019, the disclosure of which is incorporated by reference herein. In the event that the operator is also using an endoscope, the endoscopic image may also be provided on display screen (114).

The images provided through display screen (114) may help guide the operator in maneuvering and otherwise manipulating instruments within the patient's head. When used in a dilation instrument assembly (not shown), navigation guidewire (130) may facilitate navigation of instrumentation of the dilation instrument assembly within the patient during performance of a procedure to dilate the ostium of a paranasal sinus; or to dilate some other anatomical passageway (e.g., within the ear, nose, or throat, etc.). It should also be understood that other components of the dilation instrument assembly may incorporate a sensor like the sensor of navigation guidewire (130), including but not limited to a dilation catheter (not shown).

II. Example of a Head-Mounted Emitter Assembly

When field generators (106) are in fixed positions relative to chair (118) during some versions of use, rather than being in fixed positions relative to head (H) of patient (P), the frame of reference for IGS navigation system (100) (i.e., the electromagnetic field generated by field generators (106)) does not move with the head (H) of the patient (P). In some instances, a procedure may involve intentional or inadvertent movements by the patient (P) while situated in chair (118), such that the head (H) of the patient (P) may shift position, location, and/or orientation in relation to frame (104). When a navigation guidewire (130) (or other instrument having a position sensor compatible with IGS navigation system (100)) is disposed in the head (H) of the patient (P), IGS navigation system (100) may not be able to differentiate between (i) movement of navigation guidewire (130) relative to the head (H) of the patient and (ii) movement of the head (H) of the patient (P) with navigation guidewire (130) (e.g., when navigation guidewire (130) remains stationary relative to the head (H) of the patient (P) yet moves with the head (H) relative to frame (104)).

Thus, by not securing field generators (106) relative to the head (H) of the patient (P), IGS navigation system (100) may provide inaccurate position data relative to the head (H) of the patient (P) when the head (H) of the patient (P) moves while navigation guidewire (130) is disposed in the head (H) of the patient (P). Some solutions may include securing a separate position sensor to the head (H) of the patient (P) to thereby track movement of the head (H) of the patient (P) during a procedure; and to account for such tracked head (H) movement to determine the real-time position of navigation guidewire (130) (and/or some other position sensor equipped instrument) relative to the head (H) of the patient (P). However, the addition of one or more additional position sensors may be undesirable in terms of cost, procedure time, and/or other factors. In addition, even if a separate position sensor is used to track the position of the head (H) of the patient (P), having field generators (106) spaced away from the head (H) of the patient (P) may present a risk of metallic instrumentation interfering with the electromagnetic fields that are generated by field generators, which may in turn adversely affect the performance of IGS navigation system (100). It may therefore be desirable to secure field generators (106) relative to the head (H) of the patient (P) such that the frame of reference for IGS navigation system (100) moves with the head (H) of the patient (P); and such that field generators (106) are brought into close proximity with the head (H) of the patient (P).

The following description provides examples of a navigation system component in the form of an electromagnetic field emitter assembly that is configured to be secured relative to the head (H) of the patient (P), and thereby move with the head (H) of the patient (P), to improve accuracy in tracking the position of an instrument (e.g., navigation guidewire (130)) that is inserted into the head (H) of the patient (P). In particular, the electromagnetic field emitter assembly is configured to generate an electromagnetic field to serve as a frame of reference for IGS navigation system (100), and which may move together with the head (H) of the patient (P), such that the signals generated by a navigational instrument (e.g., navigation guidewire (130)) may accurately indicate the three-dimensional location of the navigational instrument within the head (H) of the patient (P) irrespective of whether or not the head (H) of the patient (P) moves in relation to a stationary object such as chair (118) and/or frame (104).

It should be understood that the electromagnetic field emitter assemblies described below may be readily incorporated into any of the various navigation systems (100) described above and in any of the various medical procedures described in the various references described herein. Other suitable ways in which the below-described electromagnetic field emitter assemblies may be used will be apparent to those skilled in the art in view of the teachings herein.

A. Example of Front-Positioned Field Emitter Assembly

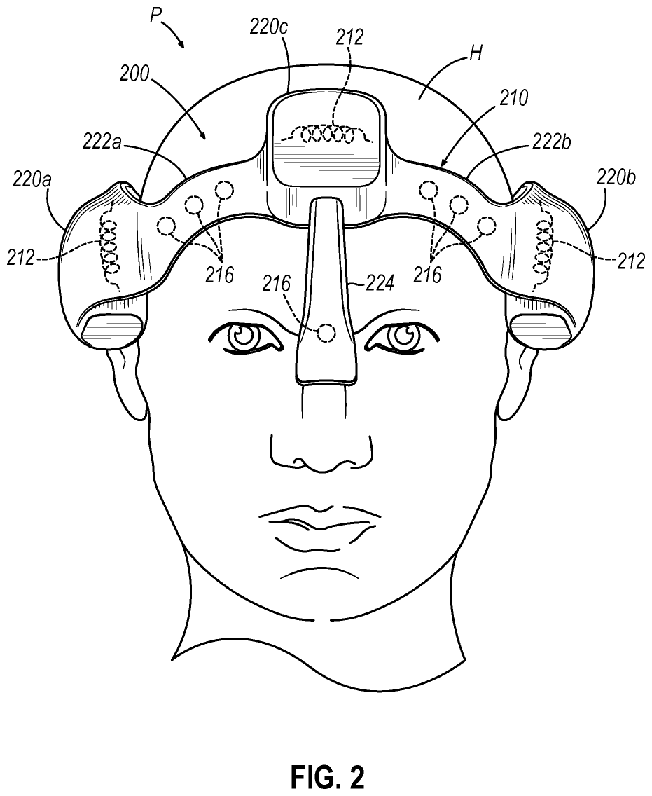

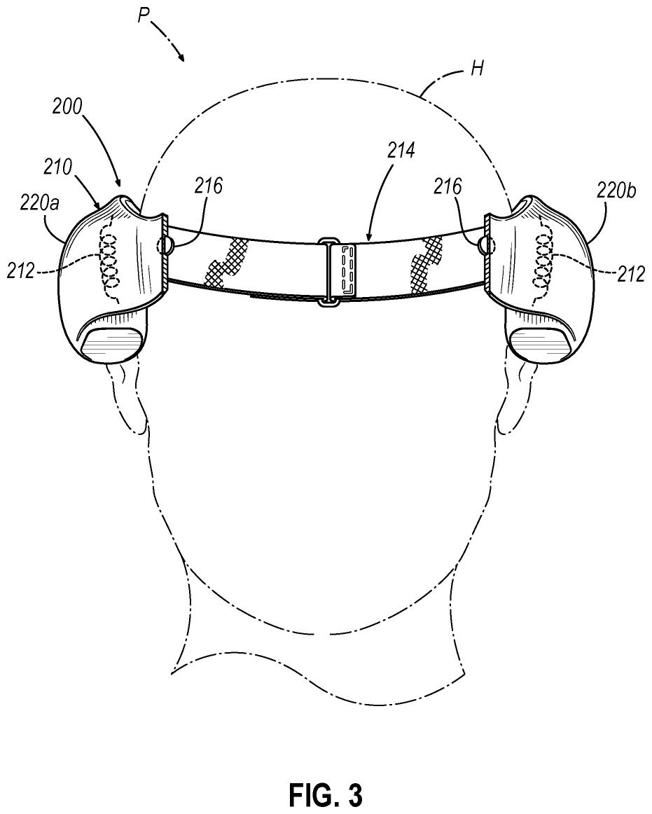

FIGS. 2-3 depict an example of an electromagnetic field emitter assembly (200) that may be readily incorporated into IGS navigation system (100), such as in place of field generator assembly (101). As will be described in greater detail below, field emitter assembly (200) comprises a frame (210), a set of magnetic field generators (212) that are integrated into frame (210), an adjustable strap (214), and a plurality of proximity sensors (216). In the present example, frame (210) is in the form of a crown; though it should be understood that frame (210) may be positioned at any suitable location and orientation on the patient's head. Field generators (212) are operable to generate alternating magnetic fields of different frequencies around the head (H) of the patient (P), and thereby enable tracking of the position of an instrument such as navigation guidewire (130) that is inserted into the head (H) of the patient (P). In some versions, field generators (212) may be operatively coupled to processor (110), such that processor (110) may be operable to drive field generators (212) to generate the electromagnetic fields. While field generators (212) are schematically shown as comprising coils in FIG. 2, various other suitable components that may be used to form and drive field generators (212) will be apparent to those skilled in the art in view of the teachings herein.

Because field generators (212) are fixedly attached to frame (210) in the present example, and because frame (210) is fixed to the head (H) of the patient (P) as described below, the electromagnetic field generated by field generators (212) is fixed against movement relative to the head (H). Therefore, signals generated by a navigational instrument (e.g., navigation guidewire (130)) may accurately indicate the three-dimensional location of the navigational instrument within the head (H) of the patient (P) irrespective of whether or not the head (H) of the patient (P) moves in relation to a stationary object such as chair (118). Processor (110) may utilize this information, such that processor (110) and display screen (114) may properly display the real-time location of navigation guidewire (130) (or any other suitable instrument) within the head (H) of the patient (P). For instance, display screen (114) may properly display the real-time location of navigation guidewire (130) (or any other suitable instrument) in a preoperative image (e.g., CT scan) of the head (H) of the patient (P), in a real-time endoscopic image obtained via an endoscope disposed in the head (H) of the patient (P), in a rendering of a 3D digital model of the head (H) of the patient (P), or otherwise.

In the present example, frame (210) includes a plurality (e.g., three) of emitter housings (220a, 220b, 220c) coupled to each other via a pair of bands (222a, 222b). More particularly, frame (210) of the present example includes a first side emitter housing (220a), a second side emitter housing (220b), and a central emitter housing (220c) coupled to first side emitter housing (220a) via a first side band (222a) and coupled to second side emitter housing (220b) via a second side band (222b). Each emitter housing (220a, 220b, 220c) securely houses or otherwise retains at least one corresponding field generator (212). In this regard, emitter housings (220a, 220b, 220c) may be positioned relative to each other such that the magnetic fields generated by the field generators (212) housed therein may collectively encompass a predefined working volume around the head (H) of the patient (P).

In the example shown, each emitter housing (220a, 220b, 220c) is configured to securely engage a corresponding portion of the head (H) of the patient (P) to inhibit movement of the field generators (212) housed therein relative to the head (H) of the patient (P) while field emitter assembly (200) is being worn by the patient (P). For example, first side emitter housing (220a) may be configured to firmly grip (e.g., press against) or otherwise securely engage a first side region of the head (H) of the patient (P), such as a first temporal region of the head (H) of the patient (P); second side emitter housing (220b) may be configured to firmly grip (e.g., press against) or otherwise securely engage a second side region of the head (H) of the patient (P), such as a second temporal region of the head (H) of the patient (P); and central emitter housing (220c) may be configured to firmly grip (e.g., press against) or otherwise securely engage a central region of the head (H) of the patient (P), such as a forehead region of the head (H) of the patient (P). In this regard, each emitter housing (220a, 220b, 220c) may include at least one head-gripping surface, which may be sized and configured to maximize contact between each head-gripping surface and the corresponding portion of the head (H) of the patient (P). For example, each head-gripping surface may be sized and shaped to complement the corresponding portion of the head (H) of the patient (P). Each head-gripping surface may also include elastomeric material and/or other feature(s) to resist movement of emitter housings (220a, 220b, 220c) relative to the head (H) of the patient (P).

In some versions, bands (222a, 222b) may be configured to urge emitter housings (220a, 220b, 220c) into secure engagement with the corresponding portions of the head (H) of the patient (P). For example, bands (222a, 222b) may each comprise a resilient material, and may cooperate with each other to resiliently bias side emitter housings (220a, 220b) toward each other. In this manner, bands (222a, 222b) may permit side emitter housings (220a, 220b) to flex slightly away from each other as frame (210) is positioned onto the head (H) of the patient (P), and may then urge side emitter housings (220a, 220b) toward each other to provide a clamping action on the head (H) of the patient (P).

Frame (210) of the present example further includes a nasal bridge extension (224) extending downwardly from central emitter housing (220c). Nasal bridge extension (224) may be sized and configured to securely engage a portion of a nose, such as a nasal bridge, of the patient (P) while emitter housings (220a, 220b, 220c) are securely engaged with the corresponding portions of the head (H) of the patient (P) to thereby assist with fixing frame (210) to the head (H) of the patient (P). In this regard, nasal bridge extension (224) may include at least one nose-gripping surface, which may be sized and configured to maximize contact between the nose-gripping surface and the corresponding portion of the patient's nose. For example, the nose-gripping surface may be sized and shaped to complement the corresponding portion of the patient's nose.

Any one or more of emitter housings (220a, 220b, 220c) and/or nasal bridge extension (224) may be substantially rigid. In addition, or alternatively, emitter housings (220a, 220b, 220c) may be fixedly coupled to each other to inhibit movement of the respective head-gripping surfaces relative to each other and/or relative to the nose-gripping surface of nasal bridge extension (224). For example, bands (222a, 222b) may be completely rigid and non-deformable. In some versions, emitter housings (220a, 220b, 220c), bands (222a, 222b), and nasal bridge extension (224) may be integrally formed together with each other as a single unitary (e.g., monolithic) piece. For example, emitter housings (220a, 220b, 220c), bands (222a, 222b), and nasal bridge extension (224) may be 3D-printed with each other as a single monolithic piece. Any one or more of the head-gripping and/or nose-gripping surfaces of frame (210) may have features that promote friction with skin of the face of the patient (P) or otherwise promote gripping of frame (210) on the face of the patient (P). Such features may include knurling, grip, a high-friction surface coating, or a thin layer of elastomer, etc. Alternatively, any other suitable kind(s) of features may be used to promote friction with skin of the face of the patient (P) or otherwise promote gripping of frame (210) on the face of the patient (P). In versions that provide a thin layer of elastomer at one or more head-gripping and/or nose-gripping surface(s), such elastomer may only deform to a negligible degree (i.e., without meaningfully impacting the function of frame (210) being fixed to the head (H) of the patient (P)), such that the thin layer of elastomer does not meaningfully impact the overall rigidity of frame (210) or otherwise facilitate inadvertent movement of emitter housings (220a, 220b, 220c) relative to the head (H) of the patient (P).

As shown in FIG. 3, adjustable strap (214) of field emitter assembly (200) extends between side emitter housings (220a, 220b) and is configured to securely engage a backside of the head (H) of the patient (P). In this regard, adjustable strap (214) may be selectively tightened against the backside of the head (H) of the patient (P) to assist with fixing frame (210) to the head (H) of the patient (P); and may be selectively loosened to facilitate positioning of frame (210) onto the head (H) of the patient (P) and removal of frame (210) from the head (H) of the patient (P).

Referring again to FIG. 2, proximity sensors (216) of field emitter assembly (200) are integrated into frame (210) at various predetermined locations. In the example shown, three proximity sensors (216) are securely housed within or otherwise retained by each band (222a, 222b) and spaced apart from each other along the length of the respective band (222a, 222b), and a single proximity sensor (216) is securely housed within or otherwise retained by nasal bridge extension (224) at or near a lower end thereof. Each proximity sensor (216) may be configured to detect a proximity of the proximity sensor (216) to the skull of the head (H) of the patient (P). For example, each proximity sensor (216) may include an infrared (IR) sensor. Proximity sensors (216) may each be in operative communication with processor (110) for providing signals to processor (110) that are indicative of the detected proximities. For example, one or more cables (not shown) may provide a conduit for communication between each proximity sensor (216) and processor (110) during some versions of a use. Alternatively, proximity sensors (216) may be connected with a wireless communication device that is in wireless communication with console (116), similar to how coupling unit (132) establishes wireless communication between navigation guidewire (130) and console (116). It will be appreciated that proximity sensors (216) may communicate with processor (110) in any other suitable manner. Processor (110) may be configured to determine a position of frame (210) relative to the skull of the head (H) of the patient (P) based on the signals received from proximity sensors (216), and/or to optimize the registration between the predefined working volume around the head (H) of the patient (P) and preoperative image(s).

In one example, a method of performing a surgical (e.g., ENT) procedure begins with an image acquisition step at which a processor, such as processor (110), acquires one or more anatomical image(s) of the head (H) of the patient (P), such as such as computerized tomography (CT), cone beam computed tomography (CBCT), or magnetic resonance imaging (MRI) image(s). In some versions, the image(s) is/are acquired as part of the ENT procedure itself (e.g., in the same “sitting” as the rest of the ENT procedure). In some other versions, the image(s) is/are acquired substantially preoperatively, such as during a procedure that is executed hours, days, weeks, or months, etc., before the rest of the ENT procedure is performed. In such scenarios, processor (110) may receive such preoperative images in any suitable fashion, including but not limited to via wired transmission, via wireless transmission, via removable storage media, etc.

Regardless of how or when processor (110) acquires the anatomical images, and regardless of when those anatomical images have been captured, the method of performing the surgical procedure may proceed with a fitting step. As part of the fitting step, field emitter assembly (200) is fitted over and fixed to the head (H) of the patient (P), such as with emitter housings (220a, 220b, 220c) securely engaging the corresponding portions of the head (H) of the patient (P), nasal bridge extension (224) securely engaging the nasal bridge of patient (P), and/or adjustable strap (214) securely engaging the backside of the head (H) of the patient (P).

Once field emitter assembly (200) has been secured to the head (H) of the patient (P), the method may then proceed with a registration step, at which processor (110) registers the one or more anatomical image(s) with the coordinate system of IGS navigation system (100), such as by utilizing the information from proximity sensors (216) to identify the position of the head (H) of the patient (P) in the coordinate system of IGS navigation system (100). Such proximity sensors (216) may be positioned and configured to sense the position of field emitter assembly (200) relative to certain anatomical landmarks on the head (H) of the patient (P), to thereby enable processor (110) to correlate the position of field emitter assembly (200) with the head (H) of the patient (P), which may in turn allow processor (110) to correlate the positions of field generators (212) relative to the stored anatomical image(s) of the head (H) of the patient (P). Thus, the presence of proximity sensors (216) may allow field emitter assembly (200) to provide substantially automatic registration, such that it is not necessary for the physician to use a separate registration stylus or other registration instrument to complete the registration process. Such manual registration processes may be reliant on the skill of the physician, such that the automated registration provided through proximity sensors (216) of field emitter assembly (200) may eliminate this reliance on the manual registration skills of the position.

In some versions, at least part of field emitter assembly (200) may be 3D-printed (or otherwise ad hoc manufactured) based on the unique anatomy of the patient (P) at hand. For instance, one or more features of field emitter assembly (200) that directly contact the patient (P)) may be 3D-printed (or otherwise ad hoc manufactured) based on the unique anatomy of the patient (P) at hand. This may ensure that field emitter assembly (200) engages the head (H) of the patient (P) in a predetermined position and orientation, which may in turn further promote accurate registration of field generators (212) with the preoperative image(s) of the anatomy of the patient (P). Such 3D-printed (or otherwise ad hoc manufactured) features of field emitter assembly (200) may be manufactured based on the same data that forms the preoperative image(s) of the anatomy of the patient (P). In some such variations, field emitter assembly (200) includes a reusable, universal portion and a disposable, patient-specific portion. The reusable, universal portion may include most of the components of field emitter assembly (200), such as field generators (212), etc. The disposable, patient-specific portion of field emitter assembly may simply include one or more features that directly engage one or more anatomical structures of the head (H) of the patient (P) (e.g., nasal bridge extension (224), etc.). Such disposable, patient-specific portion(s) may be 3D-printed (or otherwise ad hoc manufactured) based on the unique anatomy of the patient (P) at hand; while the reusable, universal portion is not necessarily patient-specific and may be used with various patients. In such versions, the reusable, universal portion and the disposable, patient-specific portion may be fixedly secured together for use during the procedure; and then may be decoupled from each other after the procedure is complete.

To the extent that field emitter assembly (200) includes one or more patient-specific patient engagement features that provide or promote registration, proximity sensors (216) may still be used to further refine the registration. Similarly, to the extent that a registration stylus and/or other registration instrument is used to provide registration, proximity sensors (216) may still be used to further refine the registration. In some cases, proximity sensors (216) may be configured to provide sufficient registration without the need for a registration stylus, one or more patient-specific patient engagement features of field emitter assembly (200), or some other registration means.

Returning to the method of the present example, after the registration step is complete, the method may then proceed with a surgical tool insertion step, at which the physician inserts a surgical tool, such as navigation guidewire (130) and/or any other position sensor equipped instrumentation, into the body of the patient (P), such as into an anatomical passageway within the head (H) of the patient (P) to perform a surgical intervention (e.g., balloon sinuplasty, etc.). Field generators (212) may cause the position sensor(s) in navigation guidewire (130) (and/or other position sensor equipped instrumentation) to generate signals indicating the real-time position of navigation guidewire (130) (and/or the other instrumentation) in three-dimensional space. It will therefore be appreciated that the sensor of navigation guidewire (130) cooperates with field generators (212) to allow the physician to track the position of a distal end of guidewire (130) using IGS navigation system (100). In this regard, the method may proceed to a navigation step, at which the physician navigates the distal end of guidewire (130) to a target location (e.g., nasal sinus) using the sensor of guidewire (130), whose measured position may be overlaid on the anatomical image(s) displayed by display screen (114).

In some scenarios, by fixing the frame of reference of navigation system (100) relative to the head (H) of the patient (P), processor (110) may accurately maintain the registration of the preoperative image(s) and the real-time position of the head (H) of the patient (P) with IGS navigation system (100) even when the patient (P) moves his/her head (H). Furthermore, processor (110) may continuously display the real-time position of guidewire (130) on the anatomical image(s) displayed by display screen (114), based on signals from the position sensor in guidewire (130), thus assisting the physician with conducting the surgical procedure. In some scenarios, by fixing field generators (212) to the head (H) of the patient (P) such that field generators (212) are relatively close to the distal end of guidewire (130) (or other surgical tool), at least by comparison to field generators (106) that are integrated into frame (104), the magnetic field produced by field generators (212) may be positioned farther away from any metal elements (e.g., metallic parts of chair (118)) than field generators (106) and thus may have an improved immunity to magnetic interference that may otherwise be caused by such metal elements.

In addition to the foregoing, field emitter assembly (200) may be configured and operable in accordance with at least some of the teachings of U.S. Pat. No. 10,314,658, entitled “Registration of an Anatomical Image with a Position-Tracking Coordinate System Based on Visual Proximity to Bone Tissue,” issued on Jun. 11, 2019, the disclosure of which is incorporated by reference herein, in its entirety; and/or U.S. Pub. No. 2021/0401500, entitled “Efficient Automatic Finding of Minimal Ear-Nose-Throat (ENT) Path for Probe,” published on Dec. 30, 2021, the disclosure of which is incorporated by reference herein, in its entirety.

B. Example of Side-Positioned Field Emitter Assembly

While FIG. 2 shows field emitter assembly (200) positioned at the front of the head (H) of a patient (P), there may be some scenarios where it is desirable to position a field emitter assembly at the side of the head (H) of the patient (P). For instance, a front-positioned field emitter assembly (200) may be preferable in scenarios where a position sensor equipped instrument will be inserted into the nasal cavity of the patient (P) to enter paranasal sinus cavities or other paranasal regions; while a side-positioned field emitter assembly may be preferable in scenarios where a position sensor equipped instrument will be inserted into an ear of the patient (P), or some region near the ear, to perform an operation in or near the middle ear or inner ear (e.g., to place a cochlear implant, etc.). In particular, a front-positioned field emitter assembly (200) may be as close as possible to the position sensor of a position sensor equipped instrument that is inserted into the nasal cavity of the patient (P); while a side-positioned field emitter assembly may be as close as possible to the position sensor of a position sensor equipped instrument that is inserted in or near an ear of the patient (P). Minimizing the distance between the field emitter assembly and the position sensor of a position sensor equipped instrument may provide greater accuracy and/or signal-to-noise ratio, etc., of position-indicative signals from the position sensor.

In addition, a side-positioned field emitter assembly may be preferable over a front-positioned field emitter assembly in procedures where the patient (P) will be in a side/lateral-lying position, as the side-positioned field emitter assembly may provide greater clearance for positioning one lateral side of the head (H) of the side/lateral-lying position patient (P) on a table or other structure while positioning the side-positioned field emitter assembly on the other lateral side of the head (H).

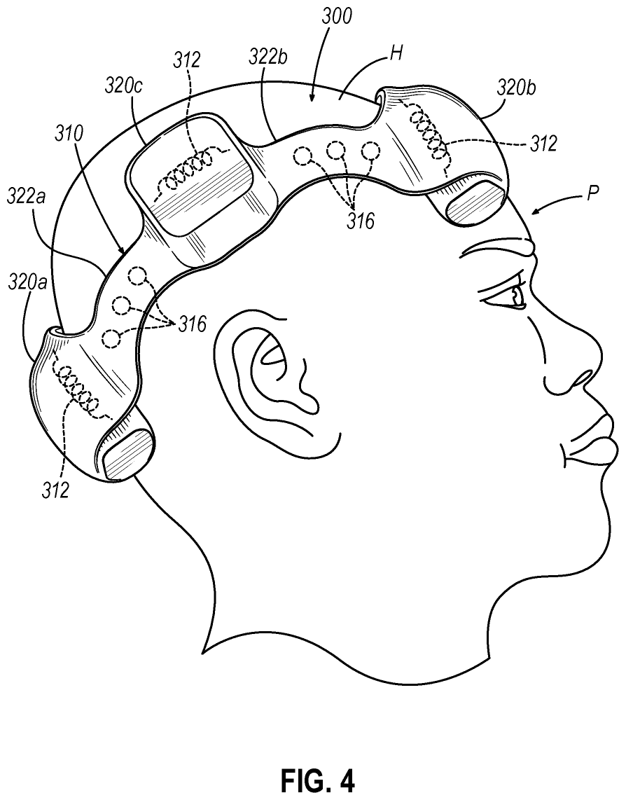

FIG. 3 shows an example of an electromagnetic field emitter assembly (300) positioned at the side of the head of a patient (P). Field emitter assembly (300) of this example is configured and operable substantially similar to field emitter assembly (200) described above. For instance, field emitter assembly (300) of this example comprises a frame (310), a set of magnetic field generators (312) that are integrated into frame (310), an adjustable strap (not shown), and a plurality of proximity sensors (316). In the present example, frame (310) is in the form of a crown; though it should be understood that frame (310) may be positioned at any suitable location and orientation on the patient's head. Field generators (312) are operable to generate alternating magnetic fields of different frequencies around the head (H) of the patient (P), and thereby enable tracking of the position of an instrument such as navigation guidewire (130) that is inserted into the head (H) of the patient (P). In some versions, field generators (312) may be operatively coupled to processor (110), such that processor (110) may be operable to drive field generators (312) to generate the electromagnetic fields. While field generators (312) are schematically shown as comprising coils in FIG. 4, various other suitable components that may be used to form and drive field generators (312) will be apparent to those skilled in the art in view of the teachings herein.

Because field generators (312) are fixedly attached to frame (310) in the present example, and because frame (310) is fixed to the head (H) of the patient (P) as described herein, the electromagnetic field generated by field generators (312) is fixed against movement relative to the head (H). Therefore, signals generated by a navigational instrument (e.g., navigation guidewire (130)) may accurately indicate the three-dimensional location of the navigational instrument within the head (H) of the patient (P) irrespective of whether or not the head (H) of the patient (P) moves in relation to a stationary object such as chair (118). Processor (110) may utilize this information, such that processor (110) and display screen (114) may properly display the real-time location of navigation guidewire (130) (or any other suitable instrument) within the head (H) of the patient (P). For instance, display screen (114) may properly display the real-time location of navigation guidewire (130) (or any other suitable instrument) in a preoperative image (e.g., CT scan) of the head (H) of the patient (P), in a real-time endoscopic image obtained via an endoscope disposed in the head (H) of the patient (P), in a rendering of a 3D digital model of the head (H) of the patient (P), or otherwise.

In the present example, frame (310) includes a plurality (e.g., three) of emitter housings (320a, 320b, 320c) coupled to each other via a pair of bands (322a, 322b). More particularly, frame (310) of the present example includes a posterior emitter housing (320a), an anterior emitter housing (320b), and a lateral emitter housing (320c). Lateral emitter housing (320c) is coupled to posterior emitter housing (320a) via a first band (322a); and to anterior emitter housing (320b) via a second band (322b). Each emitter housing (320a, 320b, 320c) securely houses or otherwise retains at least one corresponding field generator (312). In this regard, emitter housings (320a, 320b, 320c) may be positioned relative to each other such that the magnetic fields generated by the field generators (312) housed therein may collectively encompass a predefined working volume around the head (H) of the patient (P).

In the example shown, each emitter housing (320a, 320b, 320c) is configured to securely engage a corresponding portion of the head (H) of the patient (P) to inhibit movement of the field generators (312) housed therein relative to the head (H) of the patient (P) while field emitter assembly (300) is being worn by the patient (P). For example, anterior emitter housing (320a) may be configured to firmly grip (e.g., press against) or otherwise securely engage an anterior region of the head (H) of the patient (P); anterior emitter housing (320b) may be configured to firmly grip (e.g., press against) or otherwise securely engage an anterior region of the head (H) of the patient (P); and lateral emitter housing (320c) may be configured to firmly grip (e.g., press against) or otherwise securely engage a lateral region of the head (H) of the patient (P). In this regard, each emitter housing (320a, 320b, 320c) may include at least one head-gripping surface, which may be sized and configured to maximize contact between each head-gripping surface and the corresponding portion of the head (H) of the patient (P). For example, each head-gripping surface may be sized and shaped to complement the corresponding portion of the head (H) of the patient (P). Each head-gripping surface may also include elastomeric material and/or other feature(s) to resist movement of emitter housings (320a, 320b, 320c) relative to the head (H) of the patient (P).

In some versions, bands (322a, 322b) may be configured to urge emitter housings (320a, 320b, 320c) into secure engagement with the corresponding portions of the head (H) of the patient (P). For example, bands (322a, 322b) may each comprise a resilient material, and may cooperate with each other to resiliently bias side emitter housings (320a, 320b) toward each other. In this manner, bands (322a, 322b) may permit side emitter housings (320a, 320b) to flex slightly away from each other as frame (310) is positioned onto the head (H) of the patient (P), and may then urge side emitter housings (320a, 320b) toward each other to provide a clamping action on the head (H) of the patient (P).

Frame (310) of the present example does not include an extension feature that is similar to nasal bridge extension (224) of frame (210) described above. However, some variations of frame (310) may include an extension feature that is similar to nasal bridge extension (224) of frame (210) described above. For instance, such an extension feature of frame (310) may engage an ear of the patient (P) or some other anatomical structure on the head (H) of the patient. Such an extension feature is optional and may be omitted, as shown in frame (310) of FIG. 4.

Any one or more of emitter housings (320a, 320b, 320c) may be substantially rigid. In addition, or alternatively, emitter housings (320a, 320b, 320c) may be fixedly coupled to each other to inhibit movement of the respective head-gripping surfaces relative to each other. For example, bands (322a, 322b) may be completely rigid and non-deformable. In some versions, emitter housings (320a, 320b, 320c), and bands (322a, 322b) may be integrally formed together with each other as a single unitary (e.g., monolithic) piece. For example, emitter housings (320a, 320b, 320c) and bands (322a, 322b) may be 3D-printed with each other as a single monolithic piece. Any one or more of the head-gripping surfaces of frame (310) may have features that promote friction with skin of the head (H) of the patient (P) or otherwise promote gripping of frame (310) on the head (H) of the patient (P). Such features may include knurling, grip, a high-friction surface coating, or a thin layer of elastomer, etc. Alternatively, any other suitable kind(s) of features may be used to promote friction with skin of the head (H) of the patient (P) or otherwise promote gripping of frame (310) on the head (H) of the patient (P). In versions that provide a thin layer of elastomer at one or more head-gripping surface(s), such elastomer may only deform to a negligible degree (i.e., without meaningfully impacting the function of frame (310) being fixed to the head (H) of the patient (P)), such that the thin layer of elastomer does not meaningfully impact the overall rigidity of frame (310) or otherwise facilitate inadvertent movement of emitter housings (320a, 320b, 320c) relative to the head (H) of the patient (P).

While the adjustable strap of field emitter assembly (300) is not shown in FIG. 4, it should be understood that the adjustable strap of field emitter assembly (300) may be configured and operable like adjustable strap (214) of field emitter assembly (200) as described above.

Proximity sensors (316) of field emitter assembly (300) are integrated into frame (310) at various predetermined locations. In the example shown, three proximity sensors (316) are securely housed within or otherwise retained by each band (322a, 322b) and spaced apart from each other along the length of the respective band (322a, 322b). Each proximity sensor (316) may be configured to detect a proximity of the proximity sensor (316) to the skull of the head (H) of the patient (P). For example, each proximity sensor (316) may include an infrared (IR) sensor. Proximity sensors (316) may each be in operative communication with processor (110) for providing signals to processor (110) that are indicative of the detected proximities. For example, one or more cables (not shown) may provide a conduit for communication between each proximity sensor (316) and processor (110) during some versions of a use. Alternatively, proximity sensors (316) may be connected with a wireless communication device that is in wireless communication with console (116), similar to how coupling unit (132) establishes wireless communication between navigation guidewire (130) and console (116). It will be appreciated that proximity sensors (316) may communicate with processor (110) in any other suitable manner. Processor (110) may be configured to determine a position of frame (310) relative to the skull of the head (H) of the patient (P) based on the signals received from proximity sensors (316), and/or to optimize the registration between the predefined working volume around the head (H) of the patient (P) and preoperative image(s).

In one example, a method of performing a surgical procedure (e.g., cochlear implant installation procedure) begins with an image acquisition step at which a processor, such as processor (110), acquires one or more anatomical image(s) of the head (H) of the patient (P), such as such as computerized tomography (CT), cone beam computed tomography (CBCT), or magnetic resonance imaging (MRI) image(s). In some versions, the image(s) is/are acquired as part of the surgical procedure itself (e.g., in the same lateral lying position as the rest of the surgical procedure). In some other versions, the image(s) is/are acquired substantially preoperatively, such as during a procedure that is executed hours, days, weeks, or months, etc., before the rest of the surgical procedure is performed. In such scenarios, processor (110) may receive such preoperative images in any suitable fashion, including but not limited to via wired transmission, via wireless transmission, via removable storage media, etc.

Regardless of how or when processor (110) acquires the anatomical images, and regardless of when those anatomical images have been captured, the method of performing the surgical procedure may proceed with a fitting step. As part of the fitting step, field emitter assembly (300) is fitted over and fixed to the head (H) of the patient (P), such as with emitter housings (320a, 320b, 320c) and the adjustable strap securely engaging the corresponding portions of the head (H) of the patient (P).

Once field emitter assembly (300) has been secured to the head (H) of the patient (P), the method may then proceed with a registration step, at which processor (110) registers the one or more anatomical image(s) with the coordinate system of IGS navigation system (100), such as by utilizing the information from proximity sensors (316) to identify the position of the head (H) of the patient (P) in the coordinate system of IGS navigation system (100). Such proximity sensors (316) may be positioned and configured to sense the position of field emitter assembly (300) relative to certain anatomical landmarks on the head (H) of the patient (P), to thereby enable processor (110) to correlate the position of field emitter assembly (300) with the head (H) of the patient (P), which may in turn allow processor (110) to correlate the positions of field generators (312) relative to the stored anatomical image(s) of the head (H) of the patient (P). Thus, the presence of proximity sensors (316) may allow field emitter assembly (300) to provide substantially automatic registration, such that it is not necessary for the physician to use a separate registration stylus or other registration instrument to complete the registration process. Such manual registration processes may be reliant on the skill of the physician, such that the automated registration provided through proximity sensors (316) of field emitter assembly (300) may eliminate this reliance on the manual registration skills of the position.

In some versions, at least part of field emitter assembly (300) may be 3D-printed (or otherwise ad hoc manufactured) based on the unique anatomy of the patient (P) at hand. For instance, one or more features of field emitter assembly (300) that directly contact the patient (P)) may be 3D-printed (or otherwise ad hoc manufactured) based on the unique anatomy of the patient (P) at hand. This may ensure that field emitter assembly (300) engages the head (H) of the patient (P) in a predetermined position and orientation, which may in turn further promote accurate registration of field generators (312) with the preoperative image(s) of the anatomy of the patient (P). Such 3D-printed (or otherwise ad hoc manufactured) features of field emitter assembly (300) may be manufactured based on the same data that forms the preoperative image(s) of the anatomy of the patient (P). In some such variations, field emitter assembly (300) includes a reusable, universal portion and a disposable, patient-specific portion. The reusable, universal portion may include most of the components of field emitter assembly (300), such as field generators (312), etc. The disposable, patient-specific portion of field emitter assembly may simply include one or more features that directly engage one or more anatomical structures of the head (H) of the patient (P). Such disposable, patient-specific portion(s) may be 3D-printed (or otherwise ad hoc manufactured) based on the unique anatomy of the patient (P) at hand; while the reusable, universal portion is not necessarily patient-specific and may be used with various patients. In such versions, the reusable, universal portion and the disposable, patient-specific portion may be fixedly secured together for use during the procedure; and then may be decoupled from each other after the procedure is complete.

To the extent that field emitter assembly (300) includes one or more patient-specific patient engagement features that provide or promote registration, proximity sensors (316) may still be used to further refine the registration. Similarly, to the extent that a registration stylus and/or other registration instrument is used to provide registration, proximity sensors (316) may still be used to further refine the registration. In some cases, proximity sensors (316) may be configured to provide sufficient registration without the need for a registration stylus, one or more patient-specific patient engagement features of field emitter assembly (300), or some other registration means.

Returning to the method of the present example, after the registration step is complete, the method may then proceed with a surgical tool insertion step, at which the physician inserts a surgical tool, such as navigation guidewire (130) and/or any other position sensor equipped instrumentation and/or position sensor equipped implant, into the body of the patient (P), such as into an anatomical passageway within the head (H) of the patient (P) to perform a surgical intervention (e.g., installation of a cochlear implant, etc.). Field generators (312) may cause the position sensor(s) in navigation guidewire (130) (and/or other position sensor equipped instrumentation, position sensor equipped implant, etc.) to generate signals indicating the real-time position of navigation guidewire (130) (and/or the other instrumentation) in three-dimensional space. It will therefore be appreciated that the sensor of navigation guidewire (130) and/or other instrument/implant cooperates with field generators (312) to allow the physician to track the position of a distal end of guidewire (130) and/or other instrument/implant using IGS navigation system (100). In this regard, the method may proceed to a navigation step, at which the physician navigates the distal end of guidewire (130) and/or other instrument/implant to a target location (e.g., cochlea) using the sensor of guidewire (130) and/or other instrument/implant, whose measured position may be overlaid on the anatomical image(s) displayed by display screen (114).

In some scenarios, by fixing the frame of reference of navigation system (100) relative to the head (H) of the patient (P), processor (110) may accurately maintain the registration of the preoperative image(s) and the real-time position of the head (H) of the patient (P) with IGS navigation system (100) even when the patient (P) moves his/her head (H). Furthermore, processor (110) may continuously display the real-time position of guidewire (130) on the anatomical image(s) displayed by display screen (114), based on signals from the position sensor in guidewire (130) and/or other instrument/implant, thus assisting the physician with conducting the surgical procedure. In some scenarios, by fixing field generators (312) to the head (H) of the patient (P) such that field generators (312) are relatively close to the distal end of guidewire (130) and/or other instrument/implant, at least by comparison to field generators (106) that are integrated into frame (104), the magnetic field produced by field generators (312) may be positioned farther away from any metal elements (e.g., metallic parts of chair (118)) than field generators (106) and thus may have an improved immunity to magnetic interference that may otherwise be caused by such metal elements.

While the foregoing description provides a context where one field emitter assembly (200) is configured particularly for use in procedures where field emitter assembly (200) is mounted at the front/anterior region of the head (H) of the patient (P), and where another field emitter assembly (300) is configured particularly for use in procedures where field emitter assembly (300) is mounted at the side/lateral region of the head (H) of the patient (P), some other variations may provide a field emitter assembly that is configured to be selectively mounted to either the front/anterior region of the head (H) of the patient (P) or the side/lateral region of the head (H) of the patient (P), depending on the nature of the procedure at hand. In other words, there may be a “universal” version of a head-mounted field emitter assembly that is configured for use like both of the head-mounted field emitter assemblies (200, 300) described above.

III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a frame configured to be secured to a patient's head, wherein the frame includes a plurality of housings, wherein each housing of the plurality of housings is configured to securely engage a corresponding portion of the patient's head; and (b) a plurality of magnetic field generators operable to generate a magnetic field around at least a portion of the patient's head, wherein each magnetic field generator of the plurality of magnetic field generators is securely retained by a corresponding housing of the plurality of housings.

### Example 2

The apparatus of Example 1, wherein the plurality of housings includes a pair of side housings configured to securely engage corresponding temporal portions of the patient's head.

### Example 3

The apparatus of any of Examples 1 through 2, wherein the plurality of housings includes a central housing configured to securely engage a forehead portion of the patient's head.

### Example 4

The apparatus of any of Examples 1 through 3, wherein each housing of the plurality of housings is rigid.

### Example 5

The apparatus of any of Examples 1 through 4, wherein the frame further includes a nasal bridge extension, wherein the nasal bridge extension is configured to securely engage a corresponding nasal portion of the patient's head.

### Example 6

The apparatus of Example 5, wherein the nasal bridge extension is rigid.

### Example 7

The apparatus of any of Examples 1 through 6, wherein the frame further includes at least one band, wherein the at least one band couples the plurality of housings to each other.

### Example 8

The apparatus of Example 7, wherein the at least one band is rigid.

### Example 9

The apparatus of Example 7, wherein the at least one band is resiliently biased to urge the housings toward each other.

### Example 10

The apparatus of any of Examples 1 through 9, further comprising an adjustable strap coupled to the frame, wherein the adjustable strap is configured to secure the frame to the patient's head.

### Example 11

The apparatus of any of Examples 1 through 10, further comprising at least one proximity sensor secured to the frame, wherein the at least one proximity sensor is configured to detect a proximity of the at least one proximity sensor to a skull of the patient's head.

### Example 12

The apparatus of Example 11, wherein the at least one proximity sensor includes at least one infrared sensor.

### Example 13

A system comprising: (a) the apparatus of any of Examples 1 through 12; and (b) a processor, wherein the processor is configured to drive the plurality of magnetic field generators to generate the magnetic field.

### Example 14

The system of Example 13, further comprising a surgical tool, wherein the surgical tool includes a position sensor configured to generate a signal corresponding to a position of the position sensor in three-dimensional space.

### Example 15

The system of Example 14, wherein the processor is configured to receive the signal generated by the position sensor for tracking the position of the position sensor in three-dimensional space.

### Example 16

A system comprising: (a) an electromagnetic field emitter assembly including: (i) a frame configured to be secured to a patient's head, (ii) at least one magnetic field generator secured to the frame, and (iii) at least one proximity sensor secured to the frame, wherein the at least one proximity sensor is configured to generate at least one signal indicative of a proximity of the at least one proximity sensor to a skull of the patient's head; and (b) a processor, wherein the processor is configured to drive the at least one magnetic field generator to generate a magnetic field, wherein the processor is configured to receive the at least one signal from the at least one proximity sensor.

### Example 17

The system of Example 16, wherein the frame includes at least one housing configured to securely engage a corresponding portion of the patient's head, wherein the at least one magnetic field generator is securely retained by the at least one housing.

### Example 18

The system of any of Examples 16 through 17, wherein the frame includes a nasal bridge extension, wherein the nasal bridge extension is configured to securely engage a corresponding nasal portion of the patient's head, wherein the at least one proximity sensor is securely retained by the nasal bridge extension.

### Example 19

A method of performing a surgical procedure using an electromagnetic field emitter assembly, the electromagnetic field emitter assembly including: (i) a frame, and (ii) at least one magnetic field generator, the method comprising: (a) securing the frame to a patient's head, such that at least one housing of the frame securely engages a corresponding portion of the patient's head, the at least one magnetic field generator being securely retained by the at least one housing; (b) generating an electromagnetic field via the at least one magnetic field generator; and (c) navigating a surgical tool along an anatomical passageway of the patient, wherein the surgical tool includes a position sensor configured to generate a signal corresponding to a position of the position sensor in three-dimensional space.

### Example 20

The method of Example 19, further comprising performing a surgical intervention within the anatomical passageway of the patient via the surgical tool.

### Example 21

The method of any of Examples 19 through 20, the act of securing the frame to the patient's head comprising securing the frame to an anterior region of the patient's head.

### Example 22

The method of Example 21, the act of securing the frame to an anterior region of the patient's head including: (i) positioning a first portion of the frame at a first lateral region of the patient's head, (ii) positioning a second portion of the frame at a second lateral region of the patient's head, opposite to the first lateral region, and (iii) positioning a third portion of the frame along the anterior region of the patient's head, the third portion of the frame extending between the first portion of the frame and the second portion of the frame.

### Example 23

The method of any of Examples 21 through 22, the anatomical passageway of the patient comprising a nasal cavity of the patient.

### Example 24

The method of any of Examples 19 through 20, the act of securing the frame to the patient's head comprising securing the frame to a lateral region of the patient's head.

### Example 25

The method of Example 24, the act of securing the frame to a lateral region of the patient's head including: (i) positioning a first portion of the frame at an anterior region of the patient's head, (ii) positioning a second portion of the frame at a posterior region of the patient's head, and (iii) positioning a third portion of the frame along the lateral region of the patient's head, the third portion of the frame extending between the first portion of the frame and the second portion of the frame.

### Example 26

The method of any of Examples 24 through 25, the anatomical passageway of the patient being positioned at or adjacent to an ear of the patient.

### Example 27

The method of any of Examples 24 through 26, the surgical tool including a cochlear implant.

IV. Miscellaneous

As used herein, the term “tool” should be understood to include instruments, implants, and other devices that may be inserted into a body of a patient.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus comprising:
(a) a frame configured to be secured to a patient's head, wherein the frame includes a plurality of housings, wherein each housing of the plurality of housings is configured to securely engage a corresponding portion of the patient's head; and
(b) a plurality of magnetic field generators operable to generate a magnetic field around at least a portion of the patient's head, wherein each magnetic field generator of the plurality of magnetic field generators is securely retained by a corresponding housing of the plurality of housings.

2. The apparatus of claim 1, wherein the plurality of housings includes a pair of side housings configured to securely engage corresponding temporal portions of the patient's head.

3. The apparatus of claim 1, wherein the plurality of housings includes a central housing configured to securely engage a forehead portion of the patient's head.

4. The apparatus of claim 1, wherein each housing of the plurality of housings is rigid.

5. The apparatus of claim 1, wherein the frame further includes a nasal bridge extension, wherein the nasal bridge extension is configured to securely engage a corresponding nasal portion of the patient's head.

6. The apparatus of claim 5, wherein the nasal bridge extension is rigid.

7. The apparatus of claim 1, wherein the frame further includes at least one band, wherein the at least one band couples the plurality of housings to each other.

8. The apparatus of claim 7, wherein the at least one band is rigid.

9. The apparatus of claim 7, wherein the at least one band is resiliently biased to urge the housings toward each other.

10. The apparatus of claim 1, further comprising an adjustable strap coupled to the frame, wherein the adjustable strap is configured to secure the frame to the patient's head.

11. The apparatus of claim 1, further comprising at least one proximity sensor secured to the frame, wherein the at least one proximity sensor is configured to detect a proximity of the at least one proximity sensor to a skull of the patient's head.

12. The apparatus of claim 11, wherein the at least one proximity sensor includes at least one infrared sensor.

13. A system comprising:
(a) the apparatus of claim 1; and
(b) a processor, wherein the processor is configured to drive the plurality of magnetic field generators to generate the magnetic field.

14. The system of claim 13, further comprising a surgical tool, wherein the surgical tool includes a position sensor configured to generate a signal corresponding to a position of the position sensor in three-dimensional space.

15. The system of claim 14, wherein the processor is configured to receive the signal generated by the position sensor for tracking the position of the position sensor in three-dimensional space.

16. A system comprising:
(a) an electromagnetic field emitter assembly including:
(i) a frame configured to be secured to a patient's head,
(ii) at least one magnetic field generator secured to the frame, and
(iii) at least one proximity sensor secured to the frame, wherein the at least one proximity sensor is configured to generate at least one signal indicative of a proximity of the at least one proximity sensor to a skull of the patient's head; and
(b) a processor, wherein the processor is configured to drive the at least one magnetic field generator to generate a magnetic field, wherein the processor is configured to receive the at least one signal from the at least one proximity sensor.

17. The system of claim 16, wherein the frame includes at least one housing configured to securely engage a corresponding portion of the patient's head, wherein the at least one magnetic field generator is securely retained by the at least one housing.

18. The system of claim 16, wherein the frame includes a nasal bridge extension, wherein the nasal bridge extension is configured to securely engage a corresponding nasal portion of the patient's head, wherein the at least one proximity sensor is securely retained by the nasal bridge extension.

19. A method of performing a surgical procedure using an electromagnetic field emitter assembly, the electromagnetic field emitter assembly including: (i) a frame, and (ii) at least one magnetic field generator, the method comprising:
(a) securing the frame to a patient's head, such that at least one housing of the frame securely engages a corresponding portion of the patient's head, the at least one magnetic field generator being securely retained by the at least one housing;
(b) generating an electromagnetic field via the at least one magnetic field generator; and
(c) navigating a surgical tool along an anatomical passageway of the patient, wherein the surgical tool includes a position sensor configured to generate a signal corresponding to a position of the position sensor in three-dimensional space.

20. The method of claim 19, further comprising performing a surgical intervention within the anatomical passageway of the patient via the surgical tool.
